(19) United States
(12) Patent Application Publication
(10) Pub. No.: US 2006/0264677 A1
(43) Pub. Date: Nov. 23, 2006

(54) Process for the preparation of tertiary amyl hydroperoxide

(75) Inventors: Vilas Hari Rane, Pune (IN); Raj Madhukar Deshpande, Pune (IN); Raghunath Vitthal Chaudhari, Pune (IN)

Correspondence Address: LADAS & PARRY, 26 WEST 61ST STREET, NEW YORK, NY, 10023, US

(73) Assignee: COUNCIL OF SCIENTIFIC AND INDUSTRIAL RESEARCH

(21) Appl. No.: 11/375,703

(22) Filed: Mar. 14, 2006

(30) Foreign Application Priority Data
Mar. 31, 2005 (IN) 818/DEL/2005

(51) Int. Cl. C07C 409/00

(52) U.S. Cl. 568/571

(57) ABSTRACT

The present invention provides an improved process for the production of tertiary amyl hydroperoxide by the liquid phase oxidation of isopentane in presence of air or molecular oxygen as oxidant using the oxides of Group IIA metals such as magnesium, calcium, strontium and barium in high pressure reactor under stirring conditions at a temperature ranging between 110°-180° C. and at moderate pressures for a period of 0.1-12 h. The catalyst reused for sevral times without affecting its catalytic performance. The present invention produces a tertiary amyl hydroperoxide with 40-60% selectivity and tertiary amyl alcohol, which has a numerius industrial applications.

### FIELD OF INVENTION

This invention relates to an improved process for the preparation of tertiary amyl hydroperoxide. More particularly the present invention relates to a process for the production t-amyl hydroperoxide by liquid phase oxidation of isopentane or a mixture of isopentane and n-pentane in presence of air or molecular oxygen as oxidant using alkaline earth oxide catalysts.

### BACKGROUND OF THE INVENTION

Alkyl hydroperoxides are important intermediates and starting compounds for the production of valuable chemical derivatives. These can be used as a oxygen source instead of using pure oxygen. In the literature few processes have been reported for the preparation of alkyl hydroperoxides. The hydroperoxide can be prepared by reacting pure neutral dialkyl sulfate with alkali peroxide or with hydrogen peroxide in the presence of an alkali. Another method comprises reacting of monoalkyl sulfate with hydrogen peroxide followed by the neutralization of the non-aqueous portion of the reaction product and yields alkyl peroxide. Still another method comprises the production of hydroperoxides from the corresponding alcohols by treating the latter with hydrogen peroxide in the presence of certain dehydrating agents. The major drawback of these methods is the use of costly starting or intermediate material, e.g. hydrogen peroxide. Hence, one of the objectives of the present invention is to avoid the above defects and to provide an economic process for the preparation of alkyl hydroperoxide.

The interaction of alkanes—particularly branched alkanes with oxygen to produce alkyl hydroperoxides is essentially a non-catalytic reaction, but in the presence of catalysts higher yields are reported. The majority of literature available is on the formation of t-butyl hydroperoxide [TBHP] and cumene hydroperoxide [CHP]. Some literature on oxidation of isopentane to produce t-amyl hydroperoxide [TAHP] is also available.

U.S. Pat. No. 3,974,228 (1976) describes a process for the preparation of t-amyl hydroperoxide by the oxidation of isopentane in the presence of buffer comprising a basic or amphoteric compound of a metal selected from Group IIIB at 130°-150° C. and about 500-600 psig oxygen pressure. With the use of LaO, about 75% selectivity to TAHP is obtained but the conversions of isopentane are, however, poor.

U.S. Pat. No. 2,403,772 (1946) discloses a process for the production of tertiary butyl hydroperoxide (TBHP), which comprises reacting substantially equivolumetric vapors of isobutane and oxygen at superatmospheric pressure and at a temperature of about 160° C. in presence of hydrogen bromide. The yield of TBHP obtained was 75% based on oxygen consumption.

U.S. Pat. No. 2,845,461 describes a process for the production of TBHP by non-catalytic liquid phase isobutane oxidation with molecular oxygen at about 100°-150° C. and 500-700 psig pressures. Besides TBHP, tertiary butyl alcohol (TBA) is obtained as a other major side product, which has wide applications. Good isobutane conversions and a high yield to TBHP and TBA (>90%) have been reported.

U.S. Pat. NO. 5,571,908 (1996) describes TBHP formation by isobutane oxidation using porphyrin complexes of Cu, Co, Zn, Mg, but the decomposition of the TBHP was also very rapid resulting in formation of t-butanol (˜85%).

The production organic hydroperoxides by the oxidation of aryl alkyl hydrocarbons in the presence of various transition metal salt complexes has also been described in the literature. In U.S. Pat. No. 2,954,405, a process for the production of organic hydroperoxides by autooxidation of hydrocarbons in presence of molecular oxygen using metal phthalocynine as catalysts is disclosed. U.S. Pat. Nos. 5,025,101 and 5,183,945 describes a process for preparing organic hydroperoxides by selectively oxidizing aryl alkyl hydrocarbons to their corresponding organic hydroperoxides using tetranuclear manganese complexes as catalysts. U.S. Pat. No. 5,922,920 discloses the process for the production of organic hydroperoxide by oxidizing aryl alkyl hydrocarbons having a benzylic hydrogen with an oxygen containing gas using polynuclear transition metal aggregates. A process for the preparation of hydroperoxides in a homogeneous system by autooxidizing secondary alkyl group substituted methylbenzenes in the presence of water, a base and oxygen containing gas, and a water soluble chelate compound in which multidentate ligands are coordinated to at least one metal from the class of Co, Ni, Mn, Cu and Fe is disclosed in U.S. Pat. No. 4,013,725.

From the literature it can be seen that a very little work has been reported on the production of t-amyl hydroperoxide and hence there is a lot of scope for development of a process for the manufacture of TAHP. As seen from the above literature that most of the processes for the production of hydroperoxide are non-catalytic and it is advantageous to use the catalyst to increase the yield of hydroperoxide. In the present invention a solid catalyst is used for the production of TAHP by the liquid phase oxidation of isopentane, which can be reused several times without losing the activity and selectivity.

The present invention provides a process for the preparation of alkyl hydroperoxide by the liquid phase oxidation of alkanes using alkaline earth oxides as a catalyst system in the presence of air or diluted oxygen as oxidant at moderate temperatures and pressures. This invention provides a process by the use of a heterogeneous catalyst system, which can be separated from the reaction mixture with ease and reused for the another recycle experiment without affecting the catalytic performance in oxidation of isopentane.

### OBJECTIVES OF THE INVENTION

The main object of the present invention is to provide an improved process for the preparation of tertiary amyl hydroperoxide by the liquid phase oxidation of isopentane or a mixture of isopentane and n-pentane in the presence of air or molecular oxygen as oxidant using alkaline earth oxide catalysts.

Yet another object of the present invention is to provide a process for making tertiary amyl hydroperoxide with minimum by-products formation.

Yet another object of this invention is to provide catalytic liquid phase oxidation of isopentane process for the production of reaction products consisting predominantly tertiary amyl hydroperoxide and tertiary amyl alcohol.

Yet another object of this invention, air or molecular oxygen is used as an oxidant.

Yet another object of this invention is to provide a process for the production of tertiary amyl hydroperoxide at moderate temperatures and pressures.

Yet another object of this invention is to use an isopentane and n-pentane mixture for the production of tertiary amyl hydroperoxide.

### SUMMARY OF THE INVENTION

The present invention provides an improved process for the preparation of tertiary amyl hydroperoxide by the liquid phase oxidation of isopentane or a mixture of isopentane and n-pentane in presence of air or molecular oxygen as oxidant using a catalyst system consisting of oxides of alkaline earth metals. The reactions were carried out at a temperature ranging between 110°-180° C. and at moderate pressure in the presence of air or molecular oxygen as an oxidant in a high-pressure Parr reactor for a period of 0.1-12 h. After the reaction was completed, the reaction mixture was cooled to below 10° C., the reaction mixture was filtered and the reactants and products were analyzed by gas chromatograph (GC). The products were also identified by gas chromatograph—mass spectroscopy (GCMS). The present invention produces alkyl hydroperoxide with good conversion (15-20%) and selectivity (40-60%) along with tertiary amyl alcohol and other byproducts such as alcohols and ketones.

### DETAILED DESCRIPTION OF THE INVENTION

Accordingly, the present invention provides an improved process for the preparation of tertiary amyl hydroperoxide by the liquid phase oxidation of isopentane or a mixture of isopentane and n-pentane using alkaline earth oxide catalysts in the presence or absence of t-butyl hydroperoxide (TBHP) as initiator and air or molecular oxygen as an oxidant at a temperature in the range of 110-180° C. and partial pressure of oxygen in the range of 10-1000 psig for a period of 0.1-12 h, cooling the reaction mixture to 10° C., separating the catalyst and products by conventional methods.

In one of the embodiment of the present invention, the isopentane employed is neat or a mixture of isopentane and n-pentane may be in the ratio of 90:10 and 10:90.

In another embodiment the catalysts used for this invention are the oxides and carbonates of Group IIA metals i.e. magnesium, calcium, strontium and barium.

In yet another embodiment the mole ratio of the isopentane or a mixture of isopentane and n-pentane to alkaline earth metal in the oxide or carbonate catalysts is in the range of 0.5 to 200.

In yet another embodiment the mole ratio of isopentane to alkaline earth oxides or carbonates is in the range of 0.5-200.

In another embodiment the oxidant used in the reaction is air or molecular oxygen.

In yet another embodiment the reactions are carried out at a temperature in the range of 110°-180° C.

In yet another embodiment the reactions are carried out at a partial pressure of oxygen in the range of 10-1000 psig.

In still yet another embodiment the catalyst used in the said process is more economic.

In a feature of the invention the catalysts used for the process is recycled.

The process of the invention is described in detail in the following illustrative but non-limitative examples.

### EXAMPLE 1

A mixture of 37.0 g isopentane, 0.8 g TBHP and 1.0 g MgO catalyst was charged to the stirred autoclave of 300 ml capacity having a temperature and pressure controller and water condenser. The reaction vessel was heated to 140° C. and then pressurized the reactor up to 900 psig with air and continued the reaction for 2 h. The reactor was refilled with oxygen by taking into account the absorbed oxygen in the reactor and the reaction further continued for 2 h. At the end of the reaction, the reaction mixture was cooled to 10° C., filtered to separate the catalyst, and then weighed. The reactants and products were analyzed by gas chromatograph and the products were also identified by gas chromatography mass spectrometry. The GC analysis of reaction mixture showed 16.1% conversion of isopentane with 52.4% and 28.7% selectivity to t-amyl hydroperoxide and t-amyl alcohol, respectively. Ethanol, acetone and acetic acid were formed as side products with 6.5%, 12.3% and 0.2% selectivity, respectively.

### EXAMPLE 2

A mixture of 37.0 g isopentane, 0.8 g TBHP and 1.0 g MgO catalyst was charged to the stirred autoclave of 300 ml capacity having a temperature and pressure controller and water condenser. The reaction vessel was heated to 140° C. and then pressurized the reactor up to 900 psig with air and continued the reaction for 2 h. At the end of the reaction, the reaction mixture was cooled to 10° C., filtered to separate the catalyst, and then weighed. The reactants and products were analyzed by gas chromatograph and the products were also identified by gas chromatography mass spectrometry. The GC analysis of reaction mixture showed 7.0% conversion of isopentane with 61.3% and 20.4% selectivity to t-amyl hydroperoxide and t-amyl alcohol, respectively. Ethanol, acetone and acetic acid were formed as side products with 4.2%, 11.0% and 3.1% selectivity, respectively.

### EXAMPLE 3

A mixture of 37.0 g isopentane, 0.8 g TBHP and 1.0 g MgO catalyst was charged to the stirred autoclave of 300 ml capacity having a temperature and pressure controller and water condenser. The reaction vessel was heated to 140° C. and then pressurized the reactor up to 900 psig with air and continued the reaction for 2 h. The reactor was refilled with oxygen by taking into account the absorbed oxygen in the reactor and the reaction further continued for 6 h. At the end of the reaction, the reaction mixture was cooled to 10° C., filtered to separate the catalyst, and then weighed. The reactants and products were analyzed by gas chromatograph and the products were also identified by gas chromatography mass spectrometry. The GC analysis of reaction mixture showed 21.9% conversion of isopentane with 45.0% and 34.0% selectivity to t-amyl hydroperoxide and t-amyl alcohol, respectively. Ethanol, acetone and acetic acid were formed as side products with 6.5%, 12.9% and 1.7% selectivity, respectively.

### EXAMPLE 4

A mixture of 37.0 g isopentane, 0.8 g TBHP and 0.5 g MgO catalyst was charged to the stirred autoclave of 300 ml capacity having a temperature and pressure controller and water condenser. The reaction vessel was heated to 140° C. and then pressurized the reactor up to 900 psig with air and continued the reaction for 2 h. The reactor was refilled with oxygen by taking into account the absorbed oxygen in the reactor and the reaction further continued for 2 h. At the end of the reaction, the reaction mixture was cooled to 10° C., filtered to separate the catalyst, and then weighed. The reactants and products were analyzed by gas chromatograph and the products were also identified by gas chromatography mass spectrometry. The GC analysis of reaction mixture showed 19.9% conversion of isopentane with 17.8% and 44.0% selectivity to t-amyl hydroperoxide and t-amyl alcohol, respectively. Ethanol, acetone and acetic acid were formed as side products with 12.5%, 21.9% and 3.8% selectivity, respectively.

### EXAMPLE 5

A mixture of 37.0 g isopentane, 0.8 g TBHP and 2.0 g MgO catalyst was charged to the stirred autoclave of 300 ml capacity having a temperature and pressure controller and water condenser. The reaction vessel was heated to 140° C. and then pressurized the reactor up to 900 psig with air and continued the reaction for 2 h. The reactor was refilled with oxygen by taking into account the absorbed oxygen in the reactor and the reaction further continued for 2 h. At the end of the reaction, the reaction mixture was cooled to 10° C., filtered to separate the catalyst, and then weighed. The reactants and products were analyzed by gas chromatograph and the products were also identified by gas chromatography mass spectrometry. The GC analysis of reaction mixture showed 18.3% conversion of isopentane with 39.5% and 34.8% selectivity to t-amyl hydroperoxide and t-amyl alcohol, respectively. Ethanol, acetone and acetic acid were formed as side products with 8.7%, 15.8% and 1.2% selectivity, respectively.

### EXAMPLE 6

A mixture of 37.0 g isopentane, 0.8 g TBHP and 1.0 g MgO catalyst was charged to the stirred autoclave of 300 ml capacity having a temperature and pressure controller and water condenser. The reaction vessel was heated to 120° C. and then pressurized the reactor up to 900 psig with air and continued the reaction for 2 h. The reactor was refilled with oxygen by taking into account the absorbed oxygen in the reactor and the reaction further continued for 2 h. At the end of the reaction, the reaction mixture was cooled to 10° C., filtered to separate the catalyst, and then weighed. The reactants and products were analyzed by gas chromatograph and the products were also identified by gas chromatography mass spectrometry. The GC analysis of reaction mixture showed 3.3% conversion of isopentane with 74.9% and 12.5% selectivity to t-amyl hydroperoxide and t-amyl alcohol, respectively. Ethanol, acetone and acetic acid were formed as side products with 2.9%, 5.7% and 4.1% selectivity, respectively.

### EXAMPLE 7

A mixture of 37.0 g isopentane, 0.8 g TBHP and 1.0 g CaO catalyst was charged to the stirred autoclave of 300 ml capacity having a temperature and pressure controller and water condenser. The reaction vessel was heated to 140° C. and then pressurized the reactor up to 900 psig with air and continued the reaction for 2 h. The reactor was refilled with oxygen by taking into account the absorbed oxygen in the reactor and the reaction further continued for 2 h. At the end of the reaction, the reaction mixture was cooled to 10° C., filtered to separate the catalyst, and then weighed. The reactants and products were analyzed by gas chromatograph and the products were also identified by gas chromatography mass spectrometry. The GC analysis of reaction mixture showed 15.3% conversion of isopentane with 20.5% and 45.7% selectivity to t-amyl hydroperoxide and t-amyl alcohol, respectively. Ethanol, acetone and acetic acid were formed as side products with 12.3%, 21.2% and 0.3% selectivity, respectively.

### EXAMPLE 8

A mixture of 37.0 g isopentane, 0.8 g TBHP and 1.0 g CaO catalyst was charged to the stirred autoclave of 300 ml capacity having a temperature and pressure controller and water condenser. The reaction vessel was heated to 120° C. and then pressurized the reactor up to 900 psig with air and continued the reaction for 2 h. The reactor was refilled with oxygen by taking into account the absorbed oxygen in the reactor and the reaction further continued for 2 h. At the end of the reaction, the reaction mixture was cooled to 10° C., filtered to separate the catalyst, and then weighed. The reactants and products were analyzed by gas chromatograph and the products were also identified by gas chromatography mass spectrometry. The GC analysis of reaction mixture showed 3.5% conversion of isopentane with 76.1% and 11.4% selectivity to t-amyl hydroperoxide and t-amyl alcohol, respectively. Ethanol, acetone and acetic acid were formed as side products with 1.7%, 6.0% and 4.8% selectivity, respectively.

### EXAMPLE 9

A mixture of 37.0 g isopentane, 0.8 g TBHP and 1.0 g BaO catalyst was charged to the stirred autoclave of 300 ml capacity having a temperature and pressure controller and water condenser. The reaction vessel was heated to 140° C. and then pressurized the reactor up to 900 psig with air and continued the reaction for 2 h. The reactor was refilled with oxygen by taking into account the absorbed oxygen in the reactor and the reaction further continued for 2 h. At the end of the reaction, the reaction mixture was cooled to 10° C., filtered to separate the catalyst, and then weighed. The reactants and products were analyzed by gas chromatograph and the products were also identified by gas chromatography mass spectrometry. The GC analysis of reaction mixture showed 22.6% conversion of isopentane with 14.8% and 54.0% selectivity to t-amyl hydroperoxide and t-amyl alcohol, respectively. Ethanol, acetone and acetic acid were formed as side products with 12.8%, 17.7% and 0.6% selectivity, respectively.

### EXAMPLE 10

A mixture of 37.0 g isopentane, 0.8 g TBHP and 1.0 g MgCO₃ catalyst was charged to the stirred autoclave of 300 ml capacity having a temperature and pressure controller and water condenser. The reaction vessel was heated to 140° C. and then pressurized the reactor up to 900 psig with air and continued the reaction for 2 h. The reactor was refilled with oxygen by taking into account the absorbed oxygen in the reactor and the reaction further continued for 2 h. At the end of the reaction, the reaction mixture was cooled to 10° C., filtered to separate the catalyst, and then weighed. The reactants and products were analyzed by gas chromatograph and the products were also identified by gas chromatography mass spectrometry. The GC analysis of reaction mixture showed 15.2% conversion of isopentane with 12.5% and 44.5% selectivity to t-amyl hydroperoxide and t-amyl alcohol, respectively. Ethanol, acetone and acetic acid were formed as side products with 13.8%, 28.2% and 2.8% selectivity, respectively.

### EXAMPLE 11

A mixture of 37.0 g isopentane and 1.0 g MgO catalyst was charged to the stirred autoclave of 300 ml capacity having a temperature and pressure controller and water condenser. The reaction vessel was heated to 140° C. and then pressurized the reactor up to 900 psig with air and continued the reaction for 2 h. The reactor was refilled with oxygen by taking into account the absorbed oxygen in the reactor and the reaction further continued for 2 h. At the end of the reaction, the reaction mixture was cooled to 10° C., filtered to separate the catalyst, and then weighed. The reactants and products were analyzed by gas chromatograph and the products were also identified by gas chromatography mass spectrometry. The GC analysis of reaction mixture showed 9.2% conversion of isopentane with 60.2% and 19.9% selectivity to t-amyl hydroperoxide and t-amyl alcohol, respectively. Ethanol, acetone and acetic acid were formed as side products with 5.8%, 9.4% and 3.6% selectivity, respectively.

### EXAMPLE 12

A mixture of 18.0 g isopentane, 18.0 g n-pentane, 0.8 g TBHP and 1.0 g MgO catalyst was charged to the stirred autoclave of 300 ml capacity having a temperature and pressure controller and water condenser. The reaction vessel was heated to 140° C. and then pressurized the reactor up to 900 psig with air and continued the reaction for 2 h. The reactor was refilled with oxygen by taking into account the absorbed oxygen in the reactor and the reaction further continued for 2 h. At the end of the reaction, the reaction mixture was cooled to 10° C., filtered to separate the catalyst, and then weighed. The reactants and products were analyzed by gas chromatograph and the products were also identified by gas chromatography mass spectrometry. The GC analysis of reaction mixture showed 16.7% conversion of isopentane and about 4.0% conversion of n-pentane, with 45.5% and 15.8% selectivity to t-amyl hydroperoxide and t-amyl alcohol, respectively. Ethanol, acetone and acetic acid were formed as side products with 6.0%, 11.6% and 0.5% selectivity, respectively and 20.7% selectivity to 2-pentanol.

### Catalyst Recycle

### EXAMPLE 13

A mixture of 37.0 g isopentane, 0.8 g TBHP and 1.0 g MgO (recovered by the filtration of the reaction mixture followed by drying and calcination at 700° C. for 6 h) catalyst was charged to the stirred autoclave of 300 ml capacity having a temperature and pressure controller and water condenser. The reaction vessel was heated to 140° C. and then pressurized the reactor up to 900 psig with air and continued the reaction for 2 h. The reactor was refilled with oxygen by taking into account the absorbed oxygen in the reactor and the reaction further continued for 2 h. At the end of the reaction, the reaction mixture was cooled to 10° C., filtered to separate the catalyst, and then weighed. The reactants and products were analyzed by gas chromatograph and the products were also identified by gas chromatography mass spectrometry. The GC analysis of reaction mixture showed 16.7% conversion of isopentane with 57.2% and 25.8% selectivity to t-amyl hydroperoxide and t-amyl alcohol, respectively. Ethanol, acetone and acetic acid were formed as side products with 6.6%, 10.2% and 0.3% selectivity, respectively.

The advantages of the present invention are

1. The present invention provides an improved process for the production of tertiary amyl hydroperoxide by direct catalytic liquid phase oxidation of isopentane or a mixture of isopentane and n-pentane in presence of air or molecular oxygen as oxidant at ambient temperature and moderate pressure conditions.

2. The present invention provides a process by the use of heterogeneous catalysts system, which can be separated from the reaction mixture and reused for the reaction.

3. The present invention produces predominantly tertiary amyl hydroperoxide with 40-60% selectivity and also tertiary amyl alcohol, which has a numerous industrial applications.

4. The catalyst system reported in the present invention is very cheap and hence the process is more economic.

1. An improved process for the production of tertiary amyl hydroperoxide by the liquid phase oxidation of isopentane or a mixture of isopentane and n-pentane using alkaline earth oxide catalysts in the presence or absence of t-butyl hydroperoxide (TBHP) as initiator and air or molecular oxygen as an oxidant at a temperature in the range of 110-180° C. and partial pressure of oxygen in the range of 10-1000 psig for a period of 0.1-12 h, cooling the reaction mixture to 10° C., separating the catalyst and products by conventional methods.

2. A process according to claim 1, wherein the isopentane employed is neat or a mixture of isopentane and n-pentane may be in the ratio of 90:10 and 10:90.

3. A process according to claim 1, wherein the catalysts used for this invention are the oxides and carbonates of Group IIA metals i.e. magnesium, calcium, strontium and barium.

4. A process according to claim 1, wherein the mole ratio of the isopentane to alkaline earth oxides or carbonates is in the range of 0.5 to 200.

5. A process according to claim 1, wherein the oxidant used in the reaction is air or molecular oxygen.

6. In yet another embodiment the reactions are carried out at a temperature in the range of 110°-180° C.

7. A process according to claim 1, wherein the reactions are carried out at a partial pressure of oxygen in the range of 10-1000 psig.

8. A process according to claim 1, wherein the catalyst used in the said process is more economic.
